(19) United States
(12) Patent Application Publication
(10) Pub. No.: US 2009/0110751 A1
(43) Pub. Date: Apr. 30, 2009

(54) Compositions and Methods for Treatment of Diseases of the Foot of an Animal

(76) Inventor: Eli L. Kenneke, Manitowoc, WI (US)

Correspondence Address: WHYTE HIRSCHBOECK DUDEK S C;INTELLECTUAL PROPERTY DEPARTMENT, 555 EAST WELLS STREET, SUITE 1900, MILWAUKEE, WI, 53202, US

(21) Appl. No.: 12/258,987

(22) Filed: Oct. 27, 2008

Related U.S. Application Data
(60) Provisional application No. 60/982,494, filed on Oct. 25, 2007.

(51) Int. Cl. A61K 33/34; A61P 31/04

(52) U.S. Cl. 424/637

(57) ABSTRACT

The present invention is directed to an activator composition comprising copper sulfate, zinc sulfate, sodium chloride, and ammonium sulfate, for the activation of an anti-microbial composition. The resulting activated anti-microbial composition is used for the treatment of an infectious disease of a foot of an animal, particularly for the use of ungulates. Benefits of using the activator composition, either alone or in conjunction with other anti-microbial compositions, include the reduction of copper deposited into the environment from anti-microbial treatments, extended use of an anti-microbial composition, and maintenance of low levels of infectious diseases of the feet of animals.

### CROSS-REFERENCE TO RELATED APPLICATIONS

Not applicable

### FIELD OF THE INVENTION

The present invention generally relates to compositions and methods for the treatment of an infectious disease of a foot of an animal.

### BACKGROUND OF THE INVENTION

Diseases of the foot of animals, particularly ungulates, present major health problems that can cause tremendous economic losses. Lameness in cows cause animals to lose weight and body conditioning. In dairy cows, milk production is lowered because the animals tend to go to feed less often. While diseases of the hoof are most prevalent in dairy cows, beef cattle, goats, sheep, and horses, other hoofed animals are all susceptible to these pathogens.

Common diseases of a foot of an animal, particularly the hooves of animals include hairy heel warts, foot rot, and foot scald.

Papillomatous digital dermatitis, also referred to as Hairy Heel Warts, Footwarts, and Strawberry Foot Disease, is an inflammation of the skin around the hoof of an animal. It is most commonly found at the back of the foot between the bulbs of the heels, but the disease can spread around the foot and into the cleft between the claws. Digital dermatitis is a very painful condition resulting in lameness, and subsequent reduced food intake and fertility. In dairy cows, animals infected with the disease also have reduced milk yields. Within a given herd, the proportion of animals affect by digital dermatitis can remain at low levels of less than 10% while in others, over half of the animals of a herd can be affected. The severity of the disease also varies from farm to farm.

The precise cause of digital dermatitis is not known for sure, but it is known that the disease is infectious in origin and that spiral bacteria are found in the lesions whereas they are not normally found in healthy skin. In fact, some strains of the disease are consistently found deep in damaged skin.

Foot rot, or infectious pododermatitis, is a hoof infection that is commonly found in cattle, sheep, and goats. The disease, rots away the foot of the animal, more specifically the area between the two toes of the diseased animal. Like digital dermatitis, it is extremely painful and contagious.

In cattle, the cause of foot rot is two anaerobic bacteria, Fusobacterium necrophorum and Bacteroides melaninogenicus. Both bacteria are common to the environment that cattle live in and Fusobacterium is present in the rumen and fecal matter of the cattle. The disease is also caused by the microorganism Diechelobacter nodosus which can be found in contaminated soil. Another cause of foot rot may be high temperatures or humidity causing the skin between the hooves to crack and let the bacteria infect the foot. Because of this reason, foot rot is prevalent in the southern region of the United States, where the south's wet and humid climate makes it conducive for foot rot and other diseases of the hoof.

Foot rot is also prevalent in sheep and goats and must be controlled. For instance, in a Virginia survey, approximately 21 percent of the producers considered foot rot to be a serious health problem in their flocks of sheep. As in cattle, foot rot in sheep and goats is extremely painful, contagious, and causes significant economic loss due to decreased flock production.

Foot scald, also referred to as benign foot rot or interdigital dermatitis, is an inflammation between the toes caused by F. necrophorum. Persistent moisture on the skin between the toes can increase susceptibility to foot scald. Foot scald often precedes hoof rot. Like foot rot, foot scald outbreaks occur most often during persistent rainy weather along with high temperatures. If not treated these animals can become permanently infected. As with other infectious diseases of the hoof, foot scald causes stress to the animals and can affect weight gain, reproductive rates, and production. In addition, like other diseases of the hoof, Foot scald incurs additional costs to the producer for treatment and increased labor during an outbreak.

Introducing an infected animal into a non-contaminated herd can create herd contamination and therefore all animals must be treated.

Some methods currently used to treat diseases of the hoof include antibiotic treatment, chemical treatment, and anti-microbial and disinfectants.

While antibiotics can be used to rapidly reduce the severity of diseases such as digital dermatitis there are distinct disadvantages to using antibiotic therapy. For instance, the common and extensive use of antibiotics is likely to lead to the production of antibiotic resistant pathogens which are then not only problematic to the animals, but present a general concern to the public. In addition, treatment of herds with antibiotics are generally prohibitively expensive. Antibiotic therapy such as injections of long-acting penicillin or oxytetracycline, is effective, however, injections are costly and time consuming. Although antibiotics such as oxytetracycline can be applied directly to a lesion, this becomes impractical for very large herds. While antibiotic powders can be used for treatment, and Lincomycin/spectinomcin (LS-50) powder has proven to be successful, the powder is not readily absorbed into the hoof, and therefore may not reach all areas of infection. Again, this method of administration is not optimal for the treatment of large herds.

Chemicals have also been used to treat some of these diseases, however, caustic chemicals themselves can cause serious lameness due to chemical burns on the skin so therefore, great care must be taken in their use.

Anti-microbial agents and disinfectants are often used in the treatment of infectious diseases of the hoof. Such products often include solutions of 10 percent copper sulfate or zinc sulfate. A number of products are commercially available for this use, and while a number of modes of administration are possible, when a substantial number of animals in a herd are to be treated, the use of a footbath is a preferred option not only because of its ease of application, but because it is cost efficient.

In this process, animals stand in a solution of usually zinc or copper sulfate to allow absorption into the hoof wall. For treatment or control of the disease, a topical spray can also be used.

However, there are problems associated with footbaths that are currently used.

First, footbaths currently used leave high concentrations of copper in both the soil and in the water. This is because most dairies dump the spent hoof baths into a manure pit or a lagoon so the copper ultimately gets spread on production ground with the manure. The practice can lead to copper accumulation in the soil. It is possible that, after several years, copper can accumulate in soil to levels that become toxic to soil microbes and crops. This can slow organic matter decomposition and nutrient cycling in soil and crop production could be reduced because of direct toxic effects of copper on the plants as well as reduced soil fertility. Importantly, copper accumulation in soil and forage can become toxic to sheep, whose tolerance for copper is much lower than that of dairy cattle. Toxic levels of copper in soil is a critical issue because there is no practical way to reverse the problem.

The high levels of copper deposited in the soil and water are only amplified by the fact that the footbaths must be changed routinely during treatments of the herd because of contamination with dirt and manure, and dilution from use.

Accordingly, there is a need for compositions and methods for the treatment of infectious diseases of the feet of animals which not only exert potent anti-microbial properties, but also provide for reduction in the levels of copper required, which leads to reduced copper contamination to the soil and water and reduced costs of treatment.

### SUMMARY OF THE INVENTION

The present invention is generally directed to anti-microbial compositions for the treatment of an infectious disease of the foot of an animal. It has been discovered that an activator composition, described herein, can extend the antimicrobial effects and/or reduce the total levels of required copper in a variety of antimicrobial compositions, a variety of which are employed as animal footbaths for the treatment and prophylaxis of infectious diseases of the feet of animals.

One aspect of the invention provides an activator composition for activating an antimicrobial composition used for the treatment of an infectious disease of a foot of an animal. Such activator composition includes copper sulfate, zinc sulfate, sodium chloride, and ammonium sulfate. Generally, the activator composition extends anti-microbial effects of an antimicrobial composition and/or reduces total levels of required copper in an activated antimicrobial composition, as compared to an un-activated antimicrobial composition.

Another aspect of the invention provides an activated antimicrobial composition for the treatment of an infectious disease of a foot of an animal. Such activated antimicrobial composition includes (i) an activator composition comprising copper sulfate, zinc sulfate, sodium chloride, and ammonium sulfate; and (ii) an antimicrobial composition comprising water, sulfuric acid, copper sulfate, zinc sulfate, sodium chloride, potassium chloride, and ammonium sulfate. Generally, the activator composition activated antimicrobial composition extends anti-microbial effects of the antimicrobial composition and/or reduces total levels of required copper in the activated antimicrobial composition as compared to an un-activated antimicrobial composition.

Another aspect of the invention provides a kit for the treatment of an infectious disease of a foot of an animal. Such kit includes an activator composition and an antimicrobial composition. The activator composition of the kit, packaged in a first container, includes copper sulfate, zinc sulfate, sodium chloride, and ammonium sulfate. The antimicrobial composition of the kit, packaged in a second container, includes sulfuric acid, copper sulfate, zinc sulfate, sodium chloride, potassium chloride, ammonium sulfate, and, optionally, water. Combination of the activator composition of the kit and the antimicrobial composition of the kit results in the formation of an activated antimicrobial composition, where the activator composition extends anti-microbial effects of the antimicrobial composition and/or reduces total levels of required copper in the activated antimicrobial composition as compared to an un-activated antimicrobial composition.

Another aspect of the invention provides a method of making an activated antimicrobial composition for the treatment of an infectious disease of a foot of an animal. Such method includes combining an activator composition and a antimicrobial composition to form an activated antimicrobial composition. According to this method, the activator composition includes copper sulfate, zinc sulfate, sodium chloride, ammonium sulfate, and, optionally, water. According to this method, the antimicrobial composition includes sulfuric acid, copper sulfate, zinc sulfate, sodium chloride, potassium chloride, ammonium sulfate, and, optionally, water. Water can be included in the activator composition, the antimicrobial composition, or added to a mixture of the activator composition and the antimicrobial composition. According to this method, the activator composition extends anti-microbial effects of the antimicrobial composition and/or reduces total levels of required copper in the activated antimicrobial composition as compared to an un-activated antimicrobial composition.

Another aspect of the invention provides a method for the treatment of an infectious disease of a foot of an animal. Such method includes combining an activator composition and a antimicrobial composition to form an activated antimicrobial composition and administering the activated anti-microbial composition to the foot of an animal in need thereof. According to this method, the activator composition includes copper sulfate, zinc sulfate, sodium chloride, ammonium sulfate, and, optionally, water. According to this method, the antimicrobial composition includes sulfuric acid, copper sulfate, zinc sulfate, sodium chloride, potassium chloride, ammonium sulfate, and, optionally, water. Water can be included in the activator composition, the antimicrobial composition, or added to a mixture of the activator composition and the antimicrobial composition. According to this method, the activator composition extends anti-microbial effects of the antimicrobial composition and/or reduces total levels of required copper in the activated antimicrobial composition as compared to an un-activated antimicrobial composition.

In various embodiments of the above described aspects, the activator composition includes (a) copper sulfate in an amount of about 30% to about 50% by weight of the activator composition, (b) zinc sulfate in an amount of about 20% to about 40% by weight of the activator composition, (c) sodium chloride in an amount of about 10% to about 30% by weight of the activator composition, and (d) ammonium sulfate in an amount of about 0 to about 20% by weight of the activator composition. In some configurations, the activator composition includes (a) copper sulfate in an amount of about 40% by weight of the activator composition, (b) zinc sulfate in an amount of about 30% by weight of the activator composition, (c) sodium chloride in an amount of about 20% by weight of the activator composition, and (d) ammonium sulfate in an amount of about 10% by weight of the activator composition.

In various embodiments of the above described aspects, the copper sulfate is copper sulfate pentahydrate and/or the zinc sulfate is zinc sulfate monohydrate.

In various embodiments of the above described aspects, the antimicrobial composition includes about 60% to about 70% water; about 15% to about 25% sulfuric acid; about 5% to about 15% copper sulfate; about 1% to about 5% zinc sulfate; about 0.1% to about 1.5% sodium chloride; about 0.1% to about 1.5% potassium chloride, and at least about 0% to about 10% ammonium sulfate. In some configurations, the anti-microbial composition comprises about 69% by weight of water, about 20.5% by weight of sulfuric acid, about 5.0% by weight of copper sulfate, about 3.0% by weight of zinc sulfate, about 1.0% by weight of sodium chloride, about 1.0% by weight of potassium chloride, and about 0.5% by weight of ammonium sulfate. In some configurations, the anti-microbial composition comprises about 62.8% by weight of water, about 20.5% by weight of sulfuric acid, about 9.1% by weight of copper sulfate, about 1.2% by weight of zinc sulfate, about 0.3% by weight of sodium chloride, about 0.3% by weight of potassium chloride, and about 5.8% by weight of ammonium sulfate.

In various embodiments of the above described aspects, the activator composition reduces total levels of required copper in an activated antimicrobial composition as compared to an un-activated antimicrobial composition by at least about 20%. In some configurations, the activator composition reduces total levels of required copper in an activated antimicrobial composition as compared to an un-activated antimicrobial composition by at least about 40%, at least about 50%, at least about 60%, at least about 70%, or at least about 80%.

In various embodiments of the above described aspects, the infectious disease is hairy heel warts, foot rot, or scald.

In various embodiments of the above described aspects, the activated antimicrobial composition is administered by footbath, spray, gel, foam, or a combination thereof.

In various embodiments of the above described aspects, the animal to be treated is an ungulate. In some configurations, the ungulate is a cow, sheep, horse, or goat. Preferably, the animal is a cow.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a series of images depicting the Hygicult slides showing bacterial counts from a dairy cow study. For further details regarding methodology, see Example 1. FIG. 1A depicts a Hygicult slide showing the results of Step 1 plus the 17# Activator composition. FIG. 1B depicts the results of Step 2 plus the 17# Activator composition.

### DETAILED DESCRIPTION OF THE INVENTION

In accordance with the present invention, it has been discovered that the efficacy of the anti-microbial agents and the overall disinfectant properties of formulas commonly used to control diseases of the hoof can be extended with the addition of an Activator composition described herein.

The anti-microbial effects of inorganic metallic salts such as copper sulfate, zinc sulfate, and sodium chloride, which are active agents commonly used in footbath formulas such as Unifresh, Saniphresh, Hoof-Zink, Copper Hoof 2X, Healthy Hooves, Victory, and others, can be significantly extended with the addition of an activator composition described herein. All three of these salts, copper sulfate, zinc sulfate and sodium chloride have been used routinely in footbath programs of, for example, cows, sheep, and goats.

In various embodiments, the activator composition combines several of the inorganic metallic salts and a buffering ingredient (e.g., ammonium sulfate) to extend the anti-microbial efficacy and hoof conditioning properties associated with a single product. It has been discovered that ammonium sulfate, which acts as a buffering agent, provides extra buffering capacity for the footbath solution and, as such, expands anti-microbial efficacy.

As an example, upon dilution of a product such as the Unifresh Footbath product, active ingredients salts (e.g., copper sulfate, zinc sulfate, and sodium chloride) are diluted to low levels and their benefits can be negated quickly upon spoilage of the clean footbath with, for example, manure, urine, and/or dirt. Embodiments of the activator composition described herein (which comprises in part, ammonium sulfate) can concentrate and preserve these active ingredients, thus providing for higher levels of active ingredients in the footbath and longer lasting effects. Various embodiments of the activator composition described herein have been found to extend the anti-microbial and hoof conditioning benefits of the footbath for up to, for example, 500 or more cows. It has been discovered that although the activator composition can be used alone, additional benefits are realized when the activator composition is used in conjunction with other footbath products (such as Unifresh Footbath Concentrate, Unifresh Xtra, and Saniphresh Step 1 and Step 2) due to, at least in part, lowering of the pH, which more fully solubilizes inorganic metallic salts making them readily available in the footbath for anti-microbial benefit.

Another realized benefit of the activator composition is the maintenance of low levels of digital dermatitis (i.e., heel warts) and interdigital dermatitis (i.e., heel erosion and/or foot rot) with routine use. Like copper sulfate, the combination of the salts used in regular footbath program helps to promote hoof health. Healthy hooves translate to a reduction in hoof diseases and maintenance of a low incidence of these diseases in the herd. This in turn can prevent the tremendous economic losses discussed above, which can occur when animals are afflicted with these diseases.

In addition to improving the efficacy of anti-microbial compositions due to the various effects of the activator composition, environmental benefits can be appreciated with the present invention due to the reduction in the amount of copper used in the footbaths. With the present invention, because there is a reduction in the amount of copper in the footbath used, there is consequently a reduction in the amount of copper transferred to the soil and to the water. Specifically, in accordance with the present invention, it has been discovered that the copper sulfate usage per 50 gallon footbath can be significantly reduced. As an example, footbaths that traditionally are comprised of 25 pounds of copper sulfate pentahydrate for a 50 gallon footbath (which corresponds to 6.25 pounds of copper per 50 gallons), can be reduced by up to about 80% or more with the addition of the activator composition of the present invention. In footbath products such as Unifresh and Saniphresh, which typically comprise about 12 pounds of copper sulfate pentahydrate per 50 gallons (corresponding to about 3 pounds of copper per 50 gallons), the copper can be reduced by about 60% with the addition of the activator composition of the present invention while maintaining approximately equal or greater efficacy and duration of activity. As an example, the activator composition itself can contribute less than about 5 pounds (e.g., 4.8 pounds) of copper sulfate pentahydrate (comprising about 1.2 pounds of copper) per 50 gallon footbath to the environment.

The benefits of the present invention, which include enhanced efficacy of anti-microbial compositions, maintenance of low levels of infectious diseases, and/or reduced amounts of copper being transferred to the soil and water, make the use of the activated composition superior to other anti-microbial footbaths known in the art.

Composition

The present invention is generally directed to compositions and methods for the treatment of infectious diseases of a foot of an animal, particularly the hoof of an animal. Included is an activation composition for the enhancement of anti-microbial activity when the composition is added to other anti-microbial formulas. For example, the activator composition of the present invention can be added to a concentrated or diluted Tasker Unifresh® and/or Tasker Saniphresh® footbath product and/or or a concentrated or diluted Tasker Clear product. The activator composition can also be used alone as an anti-microbial product. The activator composition can be used in its diluted form or as a powder, prior to dilution.

In various embodiments, the activator composition and/or the activated antimicrobial compositions are bacteriostatic agents that are non-toxic and composed of FDA GRAS materials. The activated antimicrobial composition can provide antimicrobial effects against microbes including, but not limited to, Staphylococcus intermedius, Streptococcus mutans, E. coli, Staphylococcus aureus, Listeria, Salmonella, and Pseudomonas.

In various embodiments, the activator composition includes copper sulfate, zinc sulfate, sodium chloride, and ammonium sulfate, for the activation of an anti-microbial composition, where such an activated anti-microbial composition can be used for the treatment of an infectious disease of a foot of an animal. In various embodiments, the anti-microbial composition can include water, sulfuric acid of about 90% purity to about 99.9% purity, copper sulfate, zinc sulfate, sodium chloride, potassium chloride, and ammonium sulfate.

In a preferred embodiment of the present invention, the activator composition comprises copper sulfate in an amount of about 30% to about 50% by weight of the composition, zinc sulfate in an amount of about 20% to about 40% by weight of the composition, sodium chloride in an amount of about 10% to about 30% by weight of the composition, and ammonium sulfate in an amount of at least about 0 to about 20% by weight of the composition. For example, the activator composition can include copper sulfate in an amount of about 40% by weight of the composition, zinc sulfate in an amount of about 30% by weight of the composition, sodium chloride in an amount of about 20% by weight of the composition, and ammonium sulfate in an amount of about 10% by weight of the composition.

The activator composition can be a blended composition of the above described ingredients thus forming a homogenous mixture of the solids. The activator composition can be packaged in dry blended form (e.g., packaged in 10 lb-50 lb bags).

Generally, copper can be supplied in the compositions described herein in a variety of forms. For example, copper can be supplied as copper sulfate, preferably, copper sulfate pentahydrate. In various embodiments, copper sulfate pentahydrate is included in the activator composition in an amount of about 30% to about 50% by weight of the composition. For example, the copper sulfate pentahydrate of the activator composition can be in an amount of about 40% by weight of the composition.

Generally, zinc can be supplied in the compositions described herein in a variety of forms. For example, zinc can be supplied as zinc sulfate, preferably, zinc sulfate monohydrate. In various embodiments, zinc sulfate monohydrate is included in the activator composition in an amount of about 20% to about 40% by weight of the composition. For example, the zinc sulfate monohydrate of the activator composition can be in an amount of about 30% by weight of the composition.

As described above, the activator composition can activate an anti-microbial composition. Such activation can extend the anti-microbial effects of the antimicrobial composition. Such activation can reduce the pH of the antimicrobial composition. And such activation can reduce total levels of required copper in the activated antimicrobial composition, as compared to an un-activated antimicrobial composition.

The antimicrobial composition (which is activated by the activator composition) can include commercially available products for the treatment of infectious diseases of the foot of animals. For example, the antimicrobial composition can include, for example, Unifresh, Saniphresh, Hoof-Zink, Copper Hoof 2X, Healthy Hooves, Victory, and/or other footbath products. The antimicrobial composition activated by the activator composition can also include antimicrobial compositions such as Tasker Clear. The antimicrobial composition activated by the activator composition can also include the compositions described in U.S. Pat. No. 5,989,595; U.S. Pat. No. 6,242,011; U.S. Pat. No. 7,192,618, each of which are incorporated herein by reference in their entirety. The antimicrobial composition activated by the activator composition can also include the compositions described in U.S. Patent App. Pub. No. 2005/0191365; U.S. Patent App. Pub. No. 2005/0191394; U.S. Patent App. Pub. No. 2005/0191395; and U.S. Patent App. Pub. No. 2006/0228308. The antimicrobial composition activated by the activator composition can also include the compositions described in U.S. patent application Ser. No. 11/674,588; U.S. application Ser. No. 11/874,034; and U.S. application Ser. No. 11/750,223. One of skill in the art will understand that the concentrated antimicrobial composition can be diluted to a use concentration. Activation of the antimicrobial composition can occur before, during, or after dilution of the concentrated antimicrobial composition to the use solution.

An antimicrobial composition for activation by an activator composition described herein can include about 50% to about 80% water; about 12% to about 28% sulfuric acid; about 3% to about 18% copper sulfate; about 0.1% to about 10% zinc sulfate; about 0.1% to about 5% sodium chloride; about 0.1% to about 5% potassium chloride, and at least about 0% to about 20% ammonium sulfate. Preferably, an antimicrobial composition includes about 60% to about 70% water; about 15% to about 25% sulfuric acid; about 5% to about 15% copper sulfate; about 1% to about 5% zinc sulfate; about 0.1% to about 1.5% sodium chloride; about 0.1% to about 1.5% potassium chloride, and at least about 0% to about 10% ammonium sulfate.

As an example, a concentrated antimicrobial composition activated by the activator composition can include: about 50% to about 70% water, about 15% to about 25% sulfuric acid; about 5% to about 15% copper sulfate; about 1% to about 9% ammonium sulfate; about 1% to about 5% zinc sulfate; about 01% to about 1% sodium chloride; and about 0.1% to about 1% potassium chloride (e.g., Tasker Unifresh™). In a preferred embodiment, the antimicrobial composition includes 69.0% by weight of water; 20.5% by weight of 93% Sulfuric Acid; 5.0% by weight copper sulfate; 3.0% by weight zinc sulfate; 1.0% by weight sodium chloride; 1.0% by weight potassium chloride; 0.5% by weight ammonium sulfate (e.g., Tasker Unifresh™ FBV2 modified with 2.9% Tasker Clear). In another preferred embodiment, the antimicrobial composition includes 62.8% by weight water; 20.5% by weight of 93% Sulfuric Acid; 9.1% by weight copper sulfate; 5.8% by weight ammonium sulfate; 1.2% by weight zinc sulfate; 0.3% by weight sodium chloride; and 0.3% by weight potassium chloride (e.g., Unifresh™ FBV2 modified with 34.6% by weight of Tasker Clear and 45.5% by weight Tasker Blue).

As another example, a concentrated antimicrobial composition activated by the activator composition can include: about 35% to about 45% water; about 45% to about 55% sulfuric acid; and about 10% to about 20% ammonium sulfate (e.g., Tasker Saniphresh™, Step 1 footbath). In a preferred embodiment, the antimicrobial composition includes 52.2% by weight of 93% sulfuric acid; 36.2% by weight water; and 11.6% by weight ammonium sulfate. As a further example, a concentrated antimicrobial composition activated by the activator composition can include: about 50% to about 70% water; about 15% to about 25% sulfuric acid; about 5% to about 15% copper sulfate; about 1% to about 5% zinc sulfate; about 0.1% to about 1% sodium chloride; about 0.1% to about 1% potassium chloride; and about 0.1 to about 1% ammonium sulfate (e.g., Tasker Saniphresh™, Step 2 footbath).

Method of Making

Another embodiment of the present invention is a method of making an activated anti-microbial composition. For the purposes of this discussion, the activator composition and the antimicrobial composition are as described above. Generally, the activator composition, as described above, is combined with the antimicrobial composition, as described above. The activator composition can be combined with a concentrated antimicrobial composition or a use antimicrobial composition. The activator composition can be combined with the antimicrobial composition before or during contact with the foot of an animal. The activator composition can also be combined with the antimicrobial composition after the antimicrobial composition is contacted with the foot of an animal, for example by adding to a footbath containing antimicrobial composition or added directly as a powder, liquid, or gel on to an animal's foot already contacted with an antimicrobial composition. Preferably, the activator composition is combined with a concentrated antimicrobial composition and water so as to form an activated antimicrobial composition footbath prior to contact with an animals foot.

Various methods of combining the activator composition and the antimicrobial composition will be known to one skilled in the art. For example, the compositions can be mixed, heated, agitated, and the like so as to promote a homogenous mixture of the two components and/or to accelerate dissolution if necessary.

As an example of forming an activated antimicrobial composition, about 32 ounces of Tasker Saniphresh™ Step 1 and about 10 to about 15 pounds of the activator composition (e.g., in blended powder form) can be brought to about 50 gallons with water to form an activated antimicrobial composition footbath. As another example, about 25 ounces of Tasker Unifresh FBV2™ and about 10 to about 15 pounds of the activator composition (e.g., in blended powder form) can be brought to about 50 gallons with water to form an activated antimicrobial composition footbath. As another example, about 25 ounces of Tasker Saniphresh™ Step 2 and about 10 to about 15 pounds of the activator composition (e.g., in blended powder form) can be brought to about 50 gallons with water to form an activated antimicrobial composition footbath. As another example, about 32 ounces of Tasker Unifresh Xtra™ Step 1 and about 10 to about 15 pounds of the activator composition (e.g., in blended powder form) can be brought to about 50 gallons with water to form an activated antimicrobial composition footbath

Kit

In various embodiments, the present invention can also involve kits for use in the treatment of an infectious disease of a foot of an animal. Such kits can include the compositions of the present invention and, in certain embodiments, instructions for administration and/or usage. When supplied as a kit, the different components of the composition can be packaged in separate containers and admixed immediately before or during use. Such packaging of the components separately can, if desired, be presented in a pack or dispenser device which may contain one or more unit dosage forms containing the composition. Such packaging of the components separately can also, in certain instances, permit long-term storage without losing activity of the components. In various embodiments, the different components can be packaged in one composition for administration together.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

In various embodiments, the kit comprises an activator composition described herein along with an anti-microbial composition, such that the two can be combined so as to form an activated anti-microbial composition useful for the treatment of infectious diseases of the foot of an animal. For the purposes of this discussion, the activator composition and/or the antimicrobial composition can be as described above. The anti-microbial composition can include, but is not limited to commercially available and/or presently used footbath products. Footbath products that can be included in a kit along with an activator composition described herein include, but are not limited to, Unifresh, Saniphresh, Hoof-Zink, Copper Hoof 2X, Healthy Hooves, Victory, and other similar footbath products. For example, the activator composition can include copper sulfate, zinc sulfate, sodium chloride, and ammonium sulfate. The amount of activator composition and/or antimicrobial composition in the kit can be chosen so as to provide a therapeutically effective amount for the treatment of an infectious disease of a foot of an animal. The activator composition of the kit can be packaged in a suitable sealed container. The antimicrobial composition of the kit can be packaged in a suitable sealed container. The activator composition and/or the anti-microbial composition of the kit can be provided in dry form, liquid form, gel form, or some combination thereof. The activator composition and/or the anti-microbial composition of the kit can be provided in a concentrated form, a diluted form, or some combination thereof.

Method of Treatment

Another aspect of the invention is directed towards treatment of an infectious disease of a foot of an animal by administration of one or more of the compositions described herein to an animal in need thereof. The activator composition can be used to activate another footbath anti-microbial composition and the activated footbath composition then used to treat the foot of an animal. Alternatively, the activator composition can be used directly to treat the foot of an animal, or used in combination as a direct treatment and as an activator. The activator composition can be used as a liquid after dilution, a powder without further dilution, or some combination thereof.

As used herein, treatment is generally understood to encompass both prophylactic treatment as well as treatment of an existing condition. For example, an animal in need of treatment can be at risk for an infection of the foot. As another example, an animal in need of treatment can have, or be diagnosed with, an infection of the foot. Diagnosis and risk assessment for animal foot diseases discussed herein is within the skill of the art.

In various embodiments, the animal to be treated is an ungulate. An ungulate is understood to include an animal having hooves, or feet resembling hooves, or feet that are hoof-like. An ungulate is also understood to include an animal of, or belonging to, the former order Ungulata, now divided into the orders Perissodactyla and Artiodactyla and composed of hoofed mammals such as, but not limited to, a horse, a cow, a goat, a sheep, a pig, deer, an elephant, an elk, a bison, a moose, a gazelle, and an antelope. Preferably, the subject of various treatments described herein is a cow, a sheep, a horse, or a goat. More preferably, the ungulate to be treated with an activated anti-microbial composition of the present invention is a cow. Infectious diseases in the hooves of cows are most prevalent in dairy cow herds but are also problematic in beef cattle. The treatment compositions and methodologies described herein can be directed to dairy and/or beef cattle. In some embodiments, the ungulate to be treated with an activated anti-microbial composition of the present invention, or with the activator composition alone, is a goat. Research has shown that some goats (e.g., Boer goats) are more prone to foot rot than others. In some embodiments, the ungulate to be treated with an activated anti-microbial composition of the present invention, or with the activator composition alone, is a horse. In some embodiments, the ungulate to be treated with an activated anti-microbial composition of the present invention, or with the activator composition alone, is a sheep. Research has also shown that some breeds of sheep (e.g., Merino sheep) are more susceptible to foot rot and foot scald than other breeds.

The compositions and methods described herein, used alone or in combination with other known treatment compositions and modalities, can be directed to the treatment of an infectious disease of the foot of an animal. Such infectious diseases of the foot of an animal include, but are not limited to, hairy heel warts, foot rot, and/or scald. Foot rot includes, but is not limited to stable foot rot.

Generally, an animal is in need of treatment when an animal is diagnosed with, or at risk for, an infectious disease of the foot. As an example, an animal can be in need of treatment with the compositions described herein when the animal is diagnosed with hairy heel warts, foot rot, and/or scald. As another example, an animal can be in need of treatment with the compositions described herein when the animal is determined at risk for hairy heel warts, foot rot, and/or scald. Diagnosis of infectious diseases of the foot in animals is within the skill of the art. The determination of risk for an infectious disease of the foot can be according to environmental conditions, susceptibility of certain types of animals, or other factors known to one of skill in the art.

Animal hooves can be contacted with an activator composition and/or an activated anti-microbial composition by any method or apparatus suitable for applying the anti-microbial agent. For example, an activator composition or an activated anti-microbial composition resulting from the combination of the activator composition with an anti-microbial composition, can be administered by a method such as a footbath, powder, spray, gel, foam, or a combination thereof. One embodiment provides for the footbath, spray, gel or foam form of the activator composition to be administered sequentially with the activator composition in another form such as a footbath, spray, gel or foam. In another embodiment of the present invention, a footbath, spray, gel or foam form of an activated anti-microbial composition can be administered sequentially with an activated anti-microbial composition in another form such as a footbath, powder, spray, gel, or foam.

An activator composition or an activated anti-microbial composition can be administered in the same or different form as a single dose or in multiple applications. For example, a footbath may be administered weekly as a regimen or may be administered for different times as a regimen.

Various embodiments provide for the administration of an activator composition or an activated anti-microbial composition in any combination of a regimen in the control of an infectious disease of a foot of an animal. For example, for treatment and or control of a disease of the hoof of an animal, the activator composition or an activated anti-microbial composition can be administered as a footbath, administered as a powder, administered as a spray or a gel, or a combination thereof in various orders of administration. Duration of treatment can be according to a predetermined duration, which can involve multiple applications, or continued over a period of time until determined that treatment is no longer needed. An activator composition or an activated anti-microbial composition can be administered as a single dose or in multiple applications.

In various embodiments, an activator composition or an activated-anti-microbial composition is contacted with an animal's feet for a certain minimal contact time so as to provide for an anti-microbial effect. The contact time can vary with concentration of the use composition, method of applying the use composition, temperature of the use composition, the use composition itself (whether used alone or in conjunction with additional anti-microbial footbath products), and other similar factors that will be apparent to one of skill in the art. Preferably the exposure time is a time that allows at least partial absorption of the anti-microbial solution into the foot.

An activator composition or an activated anti-microbial composition described herein can be administered as a vented densified fluid composition, a spray of the agent, by immersion in the agent as in a footbath, by foam or gel treating with the agent, or the like, or any combination thereof. Contact with a spray, a foam, a gel, or by immersion in a liquid or immersion in a powder or other form of the anti-microbial compositions can be accomplished by a variety of methods known to those of skill in the art.

Preferably, an activator composition or an activated anti-microbial composition resulting from the combination of an activator composition with an anti-microbial composition, is administered as a footbath. An animal's foot can be exposed to a footbath of an activator composition or an activated anti-microbial composition by a variety of means known to the art, which include, for example, a trough. Another option application via footbath is the use of absorptive pads saturated with the composition to be used for treatment. The pad can be placed in a high traffic area that the animals must pass through, such as a gate, lane, or water trough. When animals stand to drink at the water, feet can be treated.

In various embodiments, an activator composition or an activated anti-microbial composition described herein can be administered as a spray. An activator composition or an activated anti-microbial composition can be applied using fixed or articulating nozzles, at higher pressures, varying or steady flow rates, various temperatures, and/or with or without agitation or brushes. Spraying can be accomplished by an apparatus such as a spray cabinet with stationary or moving spray nozzles. The nozzles can create a mist, vapor, or spray that contacts an animal's feet. The spray can be set up as a walk-through pen or in a holding pen.

Application of a material by spray can be accomplished, for example, using a manual spray wand application, an automatic spray of the animals moving through a gate or room or gateway or the like. Multiple spray heads to ensure complete contact or other spray means may be used. One preferred automatic spray application involves the use of a spray booth. The spray booth substantially confines the sprayed composition to within the parameter of the booth. The spray booth can include steam jets that can be used to apply the antimicrobial compositions of the invention. The spray pattern can be virtually any useful spray pattern.

In various embodiments, an activator composition or an activated anti-microbial composition described herein can be administered as a foam. The foam can be prepared, for example, by mixing foaming surfactants with the anti-microbial agent or composition. The foaming surfactants can be nonionic, anionic, or cationic in nature. Examples of useful surfactant types include, but are not limited to the following: alcohol ethoxylates, alcohol ethoxylate carboxylate, amine oxides, alkyl sulfates, alkyl ether sulfate, sulfonates, quaternary ammonium compounds, alkyl sarcosines, betaines, and alkyl amides. The foaming surfactant is typically mixed at time of use with the anti-microbial agent or composition but can be prepared in advance of the time of use. At time of use, compressed air can be injected into the mixture, and the foam can be applied to the hoofs. Thickeners can be further combined to produce a foam which may remain in contact with the infected area of the hoof for a longer period of time than a formulation without the thickener.

In various embodiments, an activator composition or an activated anti-microbial composition described herein can be administered as a gel. The animal foot can be treated with a thickened or gelled version of an activator composition or an activated anti-microbial composition. In the thickened or gelled state, the activator composition or the activated anti-microbial composition can remain in contact with the animal's hoof for longer periods of time, thus increasing the antimicrobial efficacy. The thickened or gelled solution can also better adhere to vertical surfaces and crevices in the animal's foot. The composition can be thickened or gelled using thickening agents including, but not limited to, xanthan gum, polymeric thickeners, cellulose thickeners or the like. Rod micelle forming systems such as amine oxides and anionic counter ions could also be used. The thickeners or gel forming agents can be used, for example, in the concentrated product or by mixing with the activator composition or the activated anti-microbial composition at time of use. Exemplary use levels of thickeners or gel agents can range from about 100 ppm to about 10 wt-%.

Immersing an animal's foot in a liquid or powder disinfectant, of the present invention, can be accomplished by any of a variety of methods known to those of skill in the art. For example, troughs can be used to immerse the feet. A disinfectant composition of the present invention, contained in the trough, can be agitated so as to increase the application and/or absorption of the solution into or onto the feet. Agitation can be obtained by conventional methods, including ultrasonics, aeration by bubbling air through the solution, by mechanical methods, such as strainers, paddles, brushes, pump driven liquid jets, or by combinations of these methods. The disinfection agent can be heated to increase the efficacy of the solution in killing micro-organisms. After the foot has been immersed for a time sufficient for the desired effect, the foot can be removed from the bath and the activator composition or the activated anti-microbial composition can be rinsed, drained, blotted, or evaporated off the hoof. Treatments with any of the compositions of the present invention can be applied one time or repeatedly within a short period of time (minutes or hours), or the treatments can be repeated as needed over a longer period of time (days or months)

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Kuiper Dairy Study

The effect of the Activator composition of the present invention was tested in an 8 week on-Farm trial (Kuiper Dairy, Hico, Tex.). At this trial site, 1201 animals are milked three times per day. There are ten pens, with no rotation, and free stalls. For this study, the footbath consisted of two 85 gallon concrete footbaths. Some erosion of the concrete was noted, particularly on the exit rims. There were no pre-baths. The footbaths were located in return lanes near milk stanchions.

For this study, Step 1 comprises 52.2 wt % of approximately 93% sulfuric acid, 36.2% deionized water, and 11.6% ammonium sulfate. Step 1 is produced as Tasker Clear® solution and can be modified with 2.9 wt % less ammonium sulfate to prevent sediment formation in cold weather.

For this study, Step 2 Footbath, which includes 3.4 wt % of Tasker Clear®, is comprised of 69.0 wt % of deionized water, 20.5 wt % of approximately 93% sulfuric acid, 5.0 wt % copper sulfate, 3.0 wt % zinc sulfate, 1.0 wt % sodium chloride, 1.0 wt % potassium chloride, and 0.5 wt % ammonium sulfate (referred to herein as Saniphresh).

The Pre-trial footbath protocol consisted of Saniphresh rotational products so that Monday/Tuesday 50 fluid ounces of Step 1 was used, and Wednesday/Thursday/Friday, 43 fluid ounces of Step 2 was used.

The dairy had completed an 8 week trial of Saniphresh Rotational products. The entire milking string was put through the baths without changing. There were approximately 600 cows and one shift per day.

In this trial, which was initiated on Jun. 1, 2007, the Activator composition (also referred to as Activator Powder) was substituted for copper sulfate in equal amounts. The Activator composition used was 1 bag of material per bath, and the package had been pre-measured and pre-packaged so that no worker measured the material for the particular study. The rotational schedule remained the same as the pre-trial rotational schedule. Saniphresh product amounts remained the same.

During the eight week study, five monitoring visits were made. In this study which was initiated on June 1, monitoring visits were made on the 1^{st}, 20^{th}, 21^{st}, 22^{nd}, and 23^{rd}of June. Results of the study were assessed by hoof trimmer records, observation of cows in milking parlor and free stalls, interviews with the dairy owner, interviews with the hoof trimmer, and TPC slides (Hygicult).

Results showed that the Activator composition buffered water at higher pH than copper sulfate. More Saniphresh was required to lower the pH value to target values, e.g., Step 1 of pH 1.4 and Step 2 of pH 2.0. Results showed that the ending pH values were typically higher on the Activator product than on equal amount of copper sulfate. The Step One ending pH with the Activator was 4.7. The Step One ending pH with the copper sulfate was 3.6-4.1. The Step Two ending pH with the Activator was 5.2-5.4 and the Step Two ending pH with copper sulfate was 4.5 to 5.2.

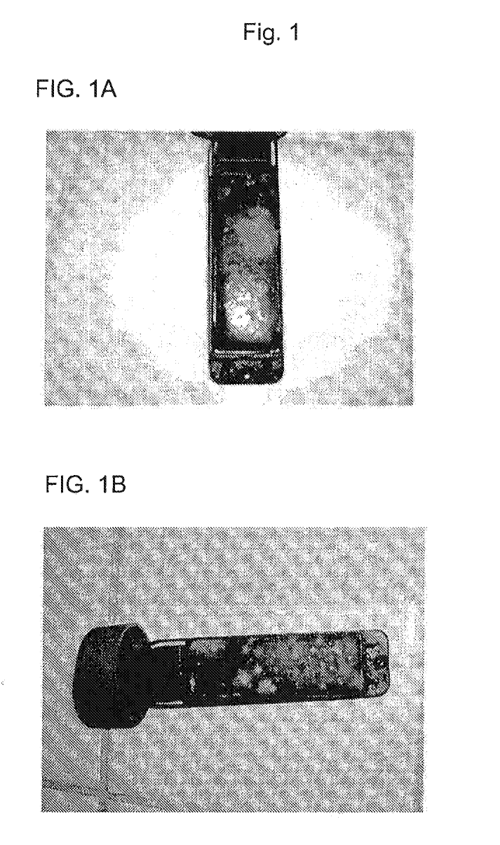

Hygicult TCP slides showed anti-microbial activity at pH values greater than 4.7 (see FIGS. 1A and 1B). Shown are photos of two Hygicult slides taken at Kuiper Dairy which was initiated June 1, 2007. FIG. 1A represents a photo of a slide from a sample taken on Jul. 22, 2007 and represents a test sample of Step 1 plus 17# Activator Powder. In this test group, 560 cows were passed through the footbath and the resulting pH of the footbath was 4.7. The bacterial count was 10₍₄₎bact./ml. FIG. 1B represents a photo of a slide from a sample taken on Jul. 23, 2007 and represents a test sample of Step 2 plus 17# Activator Powder. In this test group, 560 cows were passed through the foot bath and the resulting pH of the footbath was 5.4. The bacterial count was 10₍₃₎bact./ml.

Hooftrimmer records indicated a slight increase in the number of heel warts since the start of the Activator trial, i.e., an average of 3.2 vs. 2 warts per hooftrimmer visit (see Tables 1A, 1B and 1C). Regarding the percentage of warts per cows trimmed, there were 7% pre-trial and 11% post-trial. The largest increase was seen at the end of week 7. There was an increase in foot rot incidence since the start of the Activator trial. Specifically, there were a total of 9 cases since the start of the Activator trial, with 5% of the cows being trimmed. There were 5 cases (in 11 weeks) of foot rot while the animals were on Saniphresh plus copper sulfate, with 2% of the cows being trimmed. There were 7 cases of foot rot in the month before the Saniphresh trial, with 8% of the cows being trimmed. Several mitigating factors were noted in this study. First, there was excessive rain and muddy conditions with high humidity. Second, there was a slight increase in the number of cows trimmed per visit because of the number of cows drying off. Third, the left side of the foot bath sprang a leak on week three and was down for 10 days. Nevertheless, the hooftrimmer reported that the wart and foot rot levels were low compared to other area dairies and that the hooves were overall in good condition with the appropriate hardness. It was further noted by the hooftrimmer that there were no signs of irritation in the hooves. It is noteworthy that in this study, the customer was satisfied with the Activator product and that the customer would like to reduce copper sulfate use if the Activator composition (powder) was cost effective.

TABLE 1A
Pre-Trial Hoof Trimmer Records from the Kuiper Dairy Trial

| | # Cows | | |
|---|---|---|---|
| Date | Trimmed | # Warts | # Foot Rot |
| Feb. 10, 2007 | 26 | 0 | 6 |
| Feb. 17, 2007 | 24 | 0 | 0 |
| Feb. 23, 2007 | 14 | 0 | 0 |
| Mar. 3, 2007 | 19 | 0 | 1 |

TABLE 1B
8 Week Trial: Sanifresh Rotational Products + 17# Copper
Sulfate Start Date of Mar. 7, 2007 (Kuiper Dairy Trial)

| | # Cows | | |
|---|---|---|---|
| Date | Trimmed | # Warts | # Foot Rot |
| Mar. 10, 2007 | 17 | 17 | 0 |
| Mar. 15, 2007 | 9 | 9 | 1 |
| Mar. 24, 2007 | 37 | 37 | 1 |
| Apr. 1, 2007 | 18 | 18 | 0 |
| Apr. 7, 2007 | 35 | 35 | 2 |
| Apr. 14, 2007 | 22 | 22 | 1 |
| Apr. 20, 2007 | 17 | 17 | 3 |
| May 5, 2007 | 36 | 36 | 4 |
| May 12, 2007 | 39 | 39 | 4 |

TABLE 1C
8 Week Trial with Activator Composition (Activator Powder)
Start Date of Jun. 1, 2007 (Kuiper Dairy Trial)

| | # Cows | | |
|---|---|---|---|
| Date | Trimmed | # Warts | # Foot Rot |
| Jun. 9, 2007 | 35 | 3 | 1 |
| Jun. 16, 2007 | 20 | 5 | 3 |
| Jun. 22, 2007 | 23 | 0 | 3 |
| Jun. 30, 2007 | 21 | 1 | 0 |
| Jul. 7, 2007 | No hooftrimmer visit | | |
| Jul. 14, 2007 | 32 | 3 | 1 |
| Jul. 21, 2007 | 45 | 7 | 1 |

What is claimed is:

1. An activator composition for activating an antimicrobial composition for treatment of an infectious disease of a foot of an animal, comprising copper sulfate, zinc sulfate, sodium chloride, and ammonium sulfate;
wherein the activator composition extends anti-microbial effects of an antimicrobial composition, reduces total levels of required copper in an activated antimicrobial composition as compared to an un-activated antimicrobial composition, or both.

2. The activator composition of claim 1, comprising copper sulfate in an amount of about 30% to about 50% by weight of the activator composition, zinc sulfate in an amount of about 20% to about 40% by weight of the activator composition, sodium chloride in an amount of about 10% to about 30% by weight of the activator composition, and ammonium sulfate in an amount of about 0 to about 20% by weight of the activator composition.

3. The activator composition of claim 1, comprising copper sulfate in an amount of about 40% by weight of the activator composition, zinc sulfate in an amount of about 30% by weight of the activator composition, sodium chloride in an amount of about 20% by weight of the activator composition, and ammonium sulfate in an amount of about 10% by weight of the activator composition.

4. The activator composition of claim 1, wherein the copper sulfate comprises copper sulfate pentahydrate.

5. The activator composition of claim 1, wherein the zinc sulfate comprises zinc sulfate monohydrate.

6. An activated antimicrobial composition for the treatment of an infectious disease of a foot of an animal, comprising:
an activator composition comprising copper sulfate, zinc sulfate, sodium chloride, and ammonium sulfate; and
an antimicrobial composition;
wherein the activated antimicrobial composition is effective to reduce or prevent the infectious foot disease of the animal and extends or has a higher anti-microbial effect against a foot disease of an animal compared to the antimicrobial composition without the activator composition.

7. The activated antimicrobial composition of claim 6, wherein the antimicrobial composition comprises water and sulfuric acid.

8. The activated antimicrobial composition of claim 6, wherein the antimicrobial composition comprises water, sulfuric acid, copper sulfate, zinc sulfate, sodium chloride, potassium chloride, and ammonium sulfate.

9. The activated antimicrobial composition of claim 8, wherein the antimicrobial composition comprises about 60% to about 70% water; about 15% to about 25% sulfuric acid; about 5% to about 15% copper sulfate; about 1% to about 5% zinc sulfate; about 0.1% to about 1.5% sodium chloride; about 0.1% to about 1.5% potassium chloride, and at least about 0% to about 10% ammonium sulfate.

10. The activated antimicrobial composition of claim 8, wherein the anti-microbial composition comprises about 69% by weight of water, about 20.5% by weight of sulfuric acid, about 5.0% by weight of copper sulfate, about 3.0% by weight of zinc sulfate, about 1.0% by weight of sodium chloride, about 1.0% by weight of potassium chloride, and about 0.5% by weight of ammonium sulfate.

11. The activated antimicrobial composition of claim 8, wherein the anti-microbial composition comprises about 62.8% by weight of water, about 20.5% by weight of sulfuric acid, about 9.1% by weight of copper sulfate, about 1.2% by weight of zinc sulfate, about 0.3% by weight of sodium chloride, about 0.3% by weight of potassium chloride, and about 5.8% by weight of ammonium sulfate.

12. The activated antimicrobial composition of claim 6, wherein the activator composition reduces total levels of required copper in the activated antimicrobial composition as compared to an un-activated antimicrobial composition.

13. The activated antimicrobial composition of claim 6, wherein the activator composition reduces total levels of required copper in an activated antimicrobial composition as compared to an un-activated antimicrobial composition by at least about 20%.

14. The activated antimicrobial composition of claim 6, wherein the activator composition reduces total levels of required copper in an activated antimicrobial composition as compared to an un-activated antimicrobial composition by at least about 40%.

15. The activated antimicrobial composition of claim 6, wherein the activator composition reduces total levels of required copper in an activated antimicrobial composition as compared to an un-activated antimicrobial composition by at least about 60%.

16. The activated antimicrobial composition of claim 6, wherein the activator composition reduces total levels of required copper in an activated antimicrobial composition as compared to an un-activated antimicrobial composition by at least about 80%.

17. A kit for the treatment of an infectious disease of a foot of an animal, comprising:
an activator composition packaged in a first container, and comprising copper sulfate, zinc sulfate, sodium chloride, and ammonium sulfate; and
an antimicrobial composition packaged in a second container;
wherein the activator composition when combined with the antimicrobial composition forms an activated antimicrobial composition effective to reduce or prevent the infectious foot disease, and
the activated microbial composition extends or increases an anti-microbial effect of the antimicrobial composition compared to the antimicrobial composition without the activator composition, reduces total levels of required copper in the activated antimicrobial composition as compared to an un-activated antimicrobial composition, or both.

18. The kit of claim 17, wherein the antimicrobial composition comprises water and sulfuric acid.

19. The kit of claim 17, wherein the antimicrobial composition comprises water, sulfuric acid, copper sulfate, zinc sulfate, sodium chloride, potassium chloride, and ammonium sulfate.

20. A method of making an activated antimicrobial composition for the treatment of an infectious disease of a foot of an animal, comprising combining (a) an activator composition comprising copper sulfate, zinc sulfate, sodium chloride, and ammonium sulfate; and (b) an antimicrobial composition to form an activated antimicrobial composition;
wherein the activated composition extends or increases an anti-microbial effect of the antimicrobial composition, reduces total levels of required copper in the activated antimicrobial composition, or both, as compared to an un-activated antimicrobial composition.

21. A method for treating an infectious disease of a foot of an animal, comprising:
administering an activated anti-microbial composition to the foot of an animal in need thereof, the activated antimicrobial composition comprising (a) an activator composition comprising copper sulfate, zinc sulfate, sodium chloride, and ammonium sulfate; and (b) an antimicrobial composition.

22. The method of claim 21, wherein the infectious disease is selected from the group consisting of hairy heel warts, foot rot and scald.

23. The method of claim 21, wherein the activated antimicrobial composition is administered in a liquid form.

24. The method of claim 21, wherein the activated antimicrobial composition is administered in a footbath.

25. The method of claim 21, wherein the activated antimicrobial composition is administered in a form selected from the group consisting of a spray, gel and foam, or a combination thereof.

26. The method of claim 21, wherein the animal to be treated is an ungulate.

27. The method of claim 19, wherein the ungulate is an animal selected from the group consisting of a cow, sheep, horse and goat.

28. The method of claim 20, wherein the animal is a cow.

29. A method for treating an infectious disease of a foot of an animal, comprising:
contacting the foot of the animal with an activated antimicrobial composition comprising of a mixture of (a) an activator composition comprising copper sulfate, zinc sulfate, sodium chloride, and ammonium sulfate, and (b) an antimicrobial composition;
wherein the activated antimicrobial composition is effective to reduce or prevent the infectious foot disease of the animal, and
the activated microbial composition extends or increases an anti-microbial effect of the antimicrobial composition compared to the antimicrobial composition without the activator composition, reduces total levels of required copper in the activated antimicrobial composition as compared to the antimicrobial composition without the activator composition, or both.
